(19) United States
(12) Patent Application Publication
(10) Pub. No.: US 2025/0002445 A1
(43) Pub. Date: Jan. 2, 2025

(54) Optimized process for manufacturing methyl methacrylate

(71) Applicant: Alexander Lygin, Griesheim (DE)

(72) Inventor: Alexander Lygin, Griesheim (DE)

(21) Appl. No.: 18/420,926

(22) Filed: Jan. 24, 2024

(30) Foreign Application Priority Data
Jul. 1, 2023 (EP) 23020322

(51) Int. Cl. C07C 67/44; B01J 23/00; B01J 23/52; B01J 23/56

(52) U.S. Cl. CPC C07C 67/44; B01J 23/002; B01J 23/52; B01J 23/56; C07C 2523/52; C07C 2523/56; C07C 2523/76

(57) ABSTRACT

Process for catalytic oxidative esterification of methacrolein with methanol and oxygen to methyl methacrylate in the presence of a heterogeneous egg-shell catalyst comprising gold metal and an oxide of at least one second element selected from Ni, Co, Fe, Zn and/or Ti supported on a support material comprising SiO₂, Al₂O₃ and at least one basic element oxide, characterized in that the process is carried out in the presence of at least one compound, comprising Ni, Co, Fe, Zn and/or Ti, which is soluble in the reaction mixture under process conditions.

### FIELD OF THE INVENTION

The present invention relates to a process for producing methyl methacrylate (MMA) by catalytic oxidative esterification of methacrolein with methanol and oxygen.

### BACKGROUND OF THE INVENTION

Methyl methacrylate (MMA) is one of crucially important monomers for producing transparent poly (methyl methacrylate) and a variety of co-polymers-methacrylic polymers. There are several established processes for producing MMA, most importantly the one involving conversion of acetone cyanohydrine (ACH), but also some others e.g. those including esterification of methacrylic acid with methanol.

The so called “LIMA” process recently developed by Evonik/Röhm and described in detail in WO2014170223A1 and some other patent applications, is believed to be one of the most efficient and economical routes to MMA currently. This process consists of three major steps (1)-(3) shown below: (1) hydroformylation of ethylene to propionaldehyde, (2) amine-catalyzed condensation of propionaldehyde with formaldehyde to obtain methacrolein (MAL) and (3) direct oxidative esterification (DOE) of MAL with methanol and oxygen to yield MMA.

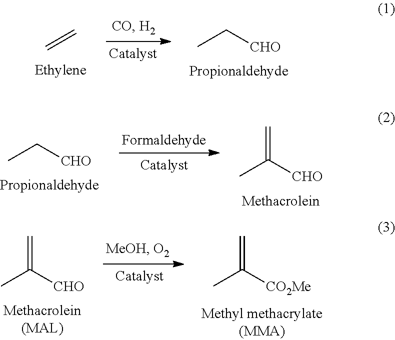

The catalytic oxidative esterification of aldehydes for preparation of carboxylic esters is described extensively in the prior art. Thus, U.S. Pat. No. 5,969,178 and U.S. Pat. No. 7,012,039 describe processes for continuously preparing MMA by direct oxidative esterification of MAL with methanol to give MMA with a Pd—Pb catalyst on an oxidic support.

US20110184206A1 describes gold and nickel oxide-containing catalysts for oxidative esterification of methacrolein to methyl methacrylate with high yield and selectivity.

U.S. Pat. Nos. 9,120,085B2 and 9,480,973B2 describe a silicon-containing material consisting of Si, Al and a basic third component such as Mg, and also a metal having elevated acid resistance as a fourth component. This fourth component is Ni, Co, Zn or Fe, distributed homogeneously in the support material. Preparing a mixture of Si, Al, the basic element and the fourth component during the production of this support material ensures such a homogeneous distribution of this fourth component throughout the whole support. This support material can be used for preparing noble metal-containing catalysts. A preferred catalyst variant for the oxidative esterification of methacrolein to MMA includes an Au/NiO catalyst supported on an SiO₂—Al₂O₃—MgO carrier material.

US2018326400A 1 discloses a catalyst for direct oxidative esterification of methacrolein, comprising silica, alumina and a basic element oxide such as MgO as well as gold and at least one oxide of iron, zinc or cobalt. This catalyst shows an egg-shell distribution of gold and (Fe, Zn or Co), wherein a maximum gold concentration or a maximum (Fe, Zn or Co) concentration of the catalyst particle in an outer region which makes up a maximum of 60% of a geometric equivalent diameter is at least 1.5 times as high as the concentration thereof in a middle region of the catalyst particle. This catalyst is described to have high productivity and selectivity to MMA, as well as low mechanical abrasion of catalyst particles and low leaching of metal ions leading to long-term retention of catalyst performance.

US20230256416A1 discloses detailed preparation of similar to those described in US2018326400A1 catalysts. Optimized catalyst preparation conditions allow reducing of gold and cobalt losses in the form of fine particles due to catalyst abrasion during the oxidative esterification process.

US20100249448A1 discloses a catalyst having an egg-shell structure with an active component comprising nickel oxide and gold nanoparticles distributed in the outer shell of the catalyst supported on a SiO₂, Al₂O₃ and a basic element (e.g. Mg) oxide. This catalyst preferably has a 0.01-15 micrometer thin outer shell substantially free of active components. A thicker than 15 μm outer shell is described to lead to decrease in catalyst activity, whereas a thinner than 0.01 μm outer shell increases abrasion of the catalyst.

Thus, change of active component distribution in the catalysts for oxidative esterification of methacrolein leads to substantial changes in long-term catalyst performance and stability. Creating a specific egg-shell distribution of the active components usually boosts catalyst performance.

However, retaining catalyst form, shape and active components distribution is often problematic in a highly dynamic environment, e.g. under intense stirring conditions in a stirred tank reactor for oxidative esterification of methacrolein. Presence of reactive components such as water, acids or alkali metal hydroxides may additionally be challenging for retaining the initial catalyst characteristics.

US20190099731A1 discloses a process for heterogeneously catalyzed reaction such as continuous oxidative esterification of methacrolein to MMA, using a reactor with two reaction zones separated by a dividing wall ensuring lower catalyst abrasion during the process. The reaction mixture flowing out of the reactor, is filtered from catalyst particles prior to its further utilization. It is suggested to use a filter with porosity 5 to 100 micrometers, preferably followed by at least one another filter with porosity of 1 to 10 micrometers for maximal retention of the catalyst in the reactor throughout the process. It is further described that the catalyst can be withdrawn from the reactor e.g. for washing or regeneration.

However, even in such a specialized reactor type, retaining catalyst form, shape and active components distribution may be challenging for the desired long process times in a continuously operated reactor.

The technical problem arising from the prior art is that of achieving continuously high yield and selectivity in the catalytic oxidative esterification of methacrolein to MMA. Particularly, it is very important to avoid changes in catalyst shape, structure and composition, which may be detrimental for catalytic performance in the process. More specifically, it is important to avoid the reducing of active metals content, particularly in the outer region of catalyst particles and thus maintain an egg-shell distribution of such active components of the catalyst for a constant and high catalyst performance. Additional problem addressed by the invention is that of increasing selectivity of the process towards formation of methyl methacrylate (MMA) and optionally methacrylic acid while decreasing the selectivity to other products such as Michael addition products of methanol to methacrolein (3-methoxy-2-methylpropanal) and to MMA (methyl 3-methoxy-2-methylpropanoate).

### BRIEF SUMMARY OF THE INVENTION

The object of the present invention is a process for catalytic oxidative esterification of methacrolein with methanol and oxygen to obtain methyl methacrylate (MMA) in the presence of a heterogeneous egg-shell catalyst comprising gold metal and an oxide of at least one second element selected from Ni, Co, Fe, Zn and/or Ti supported on a support material comprising SiO₂, Al₂O₃ and at least one basic element oxide, wherein the process is carried out in the presence of at least one compound, comprising Ni, Co, Fe, Zn and/or Ti, which is at least partially soluble in the reaction mixture under process conditions.

Present invention allows keeping the constantly high activity and selectivity of the employed egg-shell catalyst presumably by avoiding leaching of Ni, Co, Fe, Zn and/or Ti components from the outer shell of the catalyst. Without wishing to be bound by any theory, it is believed that leaching of insoluble or poorly soluble Ni, Co, Fe, Zn and/or Ti oxides from the outer shell of the egg-shell catalyst-in the form of small particles (e.g. nanoparticles) or metal ions-may be avoided or significantly decelerated by adding the corresponding metal ions into the system and thus shifting of the corresponding solubility equilibriums of poorly soluble components of the catalyst. Hence, if Ni, Co, Fe, Zn and/or Ti compounds with relatively high solubility are added to the reaction mixture, less Ni, Co, Fe, Zn and/or Ti oxides are leached from the egg-shell catalyst, particularly from the outer shell of such catalysts. This helps reserving the initial egg-shell distribution of the catalyst and thus increases high activity, selectivity, and long lifetime thereof. Another sound explanation for the retained egg-sell catalyst's distribution of Ni, Co, Fe, Zn and/or Ti in the inventive process may lay in the replenishment of these metals in the outer shell of the catalyst by re-deposition of these element from the soluble compound.

### DETAILED DESCRIPTION OF THE INVENTION

### The Egg-Shell Catalyst

The term “heterogeneous catalyst” used in the present invention is conventional and clear to the person skilled in the art and refers to a catalyst present in another phase than the reaction mixture. Specifically, the “heterogeneous catalyst” referred to in the invention is a solid-phase catalyst present in the liquid phase reaction mixture. Such heterogeneous catalyst is typically insoluble or essentially insoluble in the reaction mixture under usual reaction conditions.

The term “egg-shell catalyst” in the context of the present invention refers to a catalyst, wherein the concentration of the active components (gold and the second element oxide) in the outer region (“outer shell”) is larger than the concentration thereof in the inner region (“core”) of the catalyst.

The basic element comprised in the egg-shell catalyst may be selected from alkali metals, such as lithium (Li), sodium (Na), potassium (K); alkaline earth metals, such as magnesium (Mg), calcium (Ca), strontium (Sr), barium (Ba); rare earth metals, such as scandium (Sc), yttrium (Y), lanthanum (La), cerium (Ce), and combinations thereof. Most preferably, magnesium is used as the basic element.

The second element is selected among Ni, Co, Fe, Zn and/or Ti, preferably the second element is nickel (Ni) and/or cobalt (Co).

Most preferably, the egg-shell catalyst comprises gold, oxygen, silicon, aluminum, magnesium, cobalt and/or nickel.

The egg-shell catalyst preferably comprises, based on the total molar amount of gold, silicon, aluminum, the second element and the basic element: 0.03 mol % to 3 mol % of gold, 40 mol % to 90 mol % of silicon, 3 mol % to 40 mol % of aluminum, 0.1 mol % to 20 mol % of the second element and 2 mol % to 40 mol % of the basic element, and all these elements except for the gold and oxygen are present in the form of oxides.

The molar ratio of the second element to gold is preferably from 0.1 to 20, more preferably from 0.5 to 10, more preferably from 1 to 5.

The molar ratio of aluminum to the basic element is preferably from 0.1 to 10, more preferably from 0.3 to 5, more preferably from 0.5 to 2.

The maximum Ni, Co, Fe, Zn and/or Ti concentration in an outer region of the egg-shell catalyst particles extending from the surface of the particles to 40% of an equivalent diameter thereof is preferably at least 1.2 times, more preferably at least 1.2 times, more preferably at least 2.0 times as high as the maximum concentration of Ni, Co, Fe, Zn and/or Ti in the inner region of the egg-shell catalyst particles existing in the remaining from the outer region of the geometric equivalent diameter.

The term “equivalent diameter” used in the present invention refers to the diameter of spherical particles, or in the case of irregularly shaped particles, the diameter of a sphere having an equal volume as the particles or having a surface area equal to the surface area of the particles. Equivalent diameter is measured by measuring the mean particle diameter (volume-based) using a laser diffraction/scattering particle size distribution measuring apparatus and using the resulting value as the equivalent diameter. Alternatively, number average particle diameter as measured with a scanning electron microscope (SEM) can also be used to represent equivalent diameter.

The egg-shell catalyst preferably has a mean geometric equivalent diameter of from 10 μm to 250 μm, more preferably from 15 μm to 200 μm, more preferably from 20 μm to 150 μm, more preferably from 30 μm to 100 μm, as determined by SEM.

The thickness of the outer region in the egg-shell catalyst may be between 2 and 100 micrometers, as determined by SEM.

The egg-shell catalyst may have BET surface area of from 10 m²/g to 500 m²/g, more from 50 m²/g to 400 m²/g, more preferably from 100 m²/g to 300 m²/g. BET surface area can be determined according to DIN 9277:2014 by nitrogen adsorption in accordance with the Brunauer-Emmett-Teller method.

The egg-shell catalyst preferably has an average pore diameter of 1 nm to 50 nm. The average pore diameter can be determined by the mercury intrusion method according to DIN ISO 15901-1.

Gold is usually present in the egg-shell catalyst in the form of nanoparticles with an average diameter of below 50 nm, preferably below 20 nm, more preferably below 10 nm, more preferably from 1 nm to 10 nm. The average particle size of gold can be determined by transmission electronic microscopy (TEM). Gold can be present as individual nanoparticles and/or mixed particles comprising gold and other metals and/or metal oxides, e.g. those of Ni, Co, Fe, Zn and/or Ti.

The egg-shell catalyst preferably has a d₅₀ value of from 30 μm to 150 μm, more preferably from 40 μm to 120 μm, as determined by static light scattering (SLS) method in a 5% by weight dispersion of the catalyst in water at 25° C.

The egg-shell catalyst preferably has a d₁₀ value of at least 20 μm, more preferably at least 25 μm, more preferably at least 35 μm, as determined by static light scattering (SLS) method in a 5% by weight dispersion of the catalyst in water at 25° C.

The egg-shell catalyst preferably has a doo value of at most 300 μm, more preferably at most 200 μm, more preferably at most 150 μm, as determined by static light scattering (SLS) method in a 5% by weight dispersion of the catalyst in water at 25° C.

The values d₁₀, d₅₀ and d₉₀, reflect the particle sizes not exceeded by 10%, 50% or 90% of all particles, respectively.

### Spent Egg-Shell Catalyst

The inventive process is designed to avoid or minimize the leaching of the active components of the egg-shell catalyst, particularly the oxide of the second element. However, after a long operation time due to mechanical attrition of the particles, at least a portion of the initially employed catalyst may lose its egg-shell character and become a “spent” egg-shell catalyst.

Such a spent egg-shell catalyst, wherein the maximum Ni, Co, Fe, Zn and/or Ti concentration in an outer region of the spent egg-shell catalyst particles extending from the surface of the particles to 40% of an equivalent diameter thereof is less than 1.2 times as high as the maximum concentration of Ni, Co, Fe, Zn and/or Ti in the inner region of the spent egg-shell catalyst particles existing in the remaining from the outer region of the geometric equivalent diameter, may than at least partially be separated from the egg-shell catalyst, as defined above, and withdrawn from the reaction mixture and optionally replaced with the fresh egg-shell catalyst.

The spent egg-shell catalyst is preferably separated using filtration (i.e. smaller particles of the spent catalyst go through the filter, whereas larger particles of the egg-shell catalyst are retained in the reaction mixture) and/or sedimentation (smaller particles of the spent egg-shell catalyst are withdrawn from the upper part of the sedimentation zone, whereas the larger particles of the catalyst present in the lower part of the sedimentation zone are retained in the reaction mixture).

The spent egg-shell catalyst can be regenerated and optionally returned to the oxidative esterification process. Such a regeneration can be carried out by depositing Ni, Co, Fe, Zn and/or Ti precursors and optionally gold precursors on the outer shell of the spent egg-shell catalyst, followed by thermal decomposition of such precursors so that a fresh egg-shell catalyst is formed.

### The Compound

The compound may comprise Ni, Co, Fe, Zn and/or Ti, in elemental form, e.g. as nanoparticles, or preferably in oxidized, i.e. ionic form. Thus, the compound may comprise at least one salt of an inorganic or organic acid, such as nitrate, halide e.g. chloride, phosphate, sulphate, acetate, methacrylate and/or oxide, and/or hydroxide of the second element.

The compound comprises Ni, Co, Fe, Zn and/or Ti and is at least partially soluble in the reaction mixture under process conditions.

The term “at least partially soluble in the reaction mixture under process conditions compound” means in the context of the present invention that at least one component of the compound is at least partially soluble in said reaction mixture. Particularly, Ni, Co, Fe, Zn and/or Ti component is at least partially soluble in the mixture, i.e. Ni, Co, Fe, Zn and/or Ti can be detected in the filtered, homogeneous oxidative esterification reaction mixture comprising the compound with typical analytical methods such as inductively coupled plasma atomic emission spectroscopy (ICP-AES).

A typical procedure for determination of Ni, Co, Fe, Zn and/or Ti solubility in the reaction mixture under process conditions may look as follows: The reaction mixture produced in the process of oxidative esterification of methacrolein is removed from the reactor, filtered to remove the catalyst and cooled down to ambient temperature. A sample of thus obtained filtrate is filtered through a PTFE syringe filter having a porosity of 0.45 μm. The obtained filtrate is analyzed with the ICP-AES method to determine the content of Ni, Co, Fe, Zn and/or Ti.

Thus, the term “soluble in the reaction mixture under process conditions” does not relate to insoluble fine particles of Ni, Co, Fe, Zn and/or Ti, which may be present in the reaction mixture, e.g. due to catalyst abrasion and can be removed by a typical filtration.

Solubility of the compound may be different in different regions of the same reactor, particularly depend on the pH of the corresponding portion of the reaction mixture. The maximal solubility of the Ni, Co, Fe, Zn and/or Ti components should therefore be determined by analyzing the portion of the reaction mixture directly extracted from the region of the reaction mixture with the lowest pH level.

The solubility of the compound in the reaction mixture under process conditions is preferably so that at least 1 ppm, more preferably at least 2 ppm, more preferably at least 3 ppm, more preferably at least 5 ppm, more preferably at least 10 ppm, more preferably at least 20 ppm, more preferably at least 50 ppm, more preferably at least 100 ppm, more preferably at least 200 ppm of Ni, Co, Fe, Zn and/or Ti can be detected in the filtered, homogeneous oxidative esterification reaction mixture comprising the compound using inductively coupled plasma atomic emission spectroscopy (ICP-AES) method, wherein the filtration of the reaction mixture can be done e.g. using a filter with average porosity of at most 5 μm, more preferably at most 1 μm, e.g. 0.45 μm.

The compound and the egg-shell catalyst preferably share at least one same second element. More preferably, all second elements present in the egg-shell catalyst, are also present in the compound.

In one embodiment of the invention, the compound comprises an oxide of at least one second element selected from Ni, Co, Fe, Zn and/or Ti and optionally gold metal supported on a support material comprising SiO₂, Al₂O₃ and at least one basic element oxide such as magnesium. In this case, the compound may have a d₁₀ value of at most 15 μm, more preferably at most 12 μm, more preferably at most 10 μm, more preferably at most 7 μm, more preferably at most 5 μm, determined by static light scattering (SLS) method in a 5% by weight dispersion of the compound in water at 25° C.

In some embodiments of the invention, the egg-shell catalyst may itself be the compound showing certain solubility of the second element in the reaction mixture under process conditions. However preferably, the compound is not the egg-shell catalyst. Correspondingly, the compound preferably does not comprise gold metal.

The compound may be present in the reaction mixture prior to start of the process, e.g. added together with the egg-shell catalyst or preferably may be added, in portions or continuously, to the reaction mixture during the process.

If the process of oxidative esterification is carried out continuously, the compound is preferably added to the process continuously. In this case, the weight ratio of the mass of the compound added to the reactor per time unit to the sum of the masses of all reactants per time unit may be in the range between 0.1 ppm and 0.1 wt %, preferably from 1 ppm to 500 ppm.

The mass ratio of the compound to the egg-shell catalyst present in the reactor mixture during the process is preferably in the range 0.00001 to 1, more preferably 0.0001 to 0.1.

### The Process

### Methacrolein Synthesis

Methacrolein employed in the inventive process can preferably be obtained by a reaction of propionaldehyde with formaldehyde, i.e. the reaction according to equation (2) shown above.

This so-called Mannich reaction (2) disclosed in DE3213681A1 and elsewhere takes place in the liquid phase, in which formaldehyde and propionaldehyde are converted to methacrolein in the presence of water and in the presence of the homogeneous catalyst based at least on an acid and a base. Suitable acids are especially inorganic acids, organic monocarboxylic acids, organic dicarboxylic acids and organic polycarboxylic acids. Preference is given to using organic monocarboxylic acids such as propionic acid. Suitable bases are especially organic bases, preference being given to amines, especially secondary amines such as dimethylamine, diethylamine, methylethylamine, methylpropylamine, dipropylamine, dibutylamine, diisopropylamine, diisobutylamine, methylisopropylamine, methylisobutylamine, methyl-sec-butylamine, methyl(2-methylpentyl)amine, methyl(2-ethylhexyl)amine, pyrrolidine, piperidine, morpholine, N-methylpiperazine, N-hydroxyethylpiperazine, piperazine, hexamethyleneimine, diethanolamine, methylethanolamine, methylcyclohexylamine, methylcyclopentylamine or dicyclohexylamine. It is also possible to use mixtures of two or more bases.

### Propionaldehyde Synthesis

Propionaldehyde employed for the synthesis of methacrolein used in the inventive process can preferably be obtained by hydroformylation of ethylene, i.e. reaction according to equation (1) shown above.

The hydroformylation in general is well known, and reference is made in this connection to standard literature, for example Kirk-Othmer Encyclopedia of Chemical Technology, John Wiley & Sons, Inc., OXO Process and Franke et al., Applied Hydroformylation, dx.doi.org/10.1021/cr3001803, Chem. Rev. 2012, 112, 5675-5732. Catalysts are usually used for this reaction. Among the preferred catalysts are in particular compounds in which rhodium, iridium, palladium and/or cobalt is present, particular preference being given here to rhodium. Metal complexes which comprise at least one phosphorus-containing compound as ligand are typically used as catalysts. The hydroformylation of ethene uses carbon monoxide and hydrogen, usually in the form of a mixture known as synthesis gas. The composition of the synthesis gas used for the hydroformylation process can vary widely. The molar ratio of carbon monoxide and hydrogen is generally from 2:1 to 1:2, in particular about 45:55 to 50:50.

The temperature in the hydroformylation reaction is generally in the range of about 50 to 200° C., preferably about 60 to 190° C., in particular about 90 to 190° C. The reaction is preferably carried out at a pressure in the range of about 5 to 700 bar, preferably 10 to 200 bar, in particular 15 to 60 bar. The reaction pressure can be varied, depending on the activity of the hydroformylation catalyst used.

Most preferably, the sequence of processes according to equations (1), (2) and (3), i.e. hydroformylation of ethylene followed by the reaction of the obtained propionaldehyde with formaldehyde to methacrolein and oxidative esterification of MAL to MMA, is used in the present invention to obtain methyl methacrylate.

### Oxidative Esterification of Methacrolein

The process according to the invention may be carried out batchwise, semi-continuously or preferably continuously.

The reaction temperature during the oxidative esterification process is preferably between 60 and 100° C., more preferably between 70 and 95° C. The internal reactor pressure is preferably between 1 and 20 bar, more preferably between 2 and 10 bar.

The molar ratio of methanol to methacrolein in the reactor is preferably between 3:1 and 15:1, more preferably between 3.5:1 and 14:1, more preferably between 4:1 and 10:1, more preferably between 4:1 and 8:1.

Particular preference is given to an execution of the inventive process in which the partial oxygen pressure in the reactor, or in the individual reactors at the point at which offgas is withdrawn, is between 0.01 and 0.8 bar, and in the gas mixture fed to the reactor is less than 5 bar.

Preferably, the gas required for the reaction is metered in in the finely dispersed state via gas distributors, called spargers, in the lower reactor portion. Preferably, the gas used is metered in in the direction toward the reactor base, in order that a minimum level of blockage with the catalyst particles can occur.

Equally preferably, in addition to this or independently thereof, the steady-state methacrolein concentration in the reactor, or in the individual reactors, is between 1% and 21% by weight, more preferably between 3% and 21% by weight, most preferably between 5% and 20% by weight.

Reactor or reactors used in the inventive process are preferably slurry reactors. In the case of a serial connection of two or more reactors, it is also possible to combine various kinds of reactors with one another.

Preferably, reaction mixture is discharged continuously from the reactor and the catalyst remains in the reactor. In an illustrative embodiment, the reaction mixture is filtered through at least one filter, preferably located within the reactor. In this case, the catalyst beneficially remains in the reactor after the filtration.

Alternatively, the reaction mixture can be discharged continuously from the reactor and filtered through at least one filter, for example an external filter. Likewise preferably, the catalyst is passed back into the reactor after the filtration. For this purpose, continuously operable filters which can optionally be backflushed can be used, which are preferably within the reactor, more preferably at the periphery in the upper portion of the reactor. Thereafter, the catalyst is optionally subjected to further treatment after the filtration and passed partly or completely back into the reactor. This further treatment may, for example, involve washing, reactivating or separation by particle size.

The filter porosity used with preference is between 5 and 100 micrometers, more preferably between 10 and 50 micrometers. For additional retention of the fine catalyst particles, the reaction mixture, once it has been filtered through reactor filters, is preferably filtered at least once more through finer filters having porosity of 1 to 10 μm outside the reactor, such that the particles of not more than 5 μm are retained by the filter to an extent of at least 90%.

It is advantageous when the reaction mixture, after being withdrawn continuously from the reactor, is worked up in at least one distillation column, and the methanol and methacrolein are separated off from methyl methacrylate as distillate and returned to the reactor.

Apart from the reactants required for the reaction, various auxiliaries can be supplied to the process, for example acids, bases, polymerization inhibitors, antifoams, etc.

MMA is the main product of the inventive process obtained with a selectivity of at least 90%, preferably at least 92%, more preferably at least 93%, more preferably at least 94%. The most important side-products formed in the process include methacrylic acid and Michael addition products of methanol to methacrolein (3-methoxy-2-methylpropanal) and to MMA (methyl 3-methoxy-2-methylpropanoate) with a total selectivity of up to 7%, preferably up to 5%, more preferably up to 4%. Methacrylic acid is a valuable product as such, which may be used e.g. for producing mehacrylic (co)polymers, while 3-methoxy-2-methylpropanal methyl 3-methoxy-2-methylpropanoate typically cannot be used without further modifications. Therefore, it is advantageous to minimize the formation of these Michael addition products.

Formation of methacrylic acid in the process can lead to significant acidification of the reaction mixture and hence reducing of the pH, which in turn may lead to decrease in catalytic activity. Therefore, the process of the invention is preferably accompanied by adjusting the pH of the reaction mixture by adding a basic solution, preferably an alkali hydroxide solution such as KOH and/or NaOH to achieve the pH range between 5.5 and 9.0, more preferably 5.8 to 8.0, more preferably 6.0 to 7.5 in the reaction mixture.

Higher pH levels usually lead to higher catalytic activity. However, pH of the reaction mixture may have significant impact on the formation of side-products. Thus, at higher pH levels, the selectivity to undesired 3-methoxy-2-methylpropanal and methyl 3-methoxy-2-methylpropanoate is typically increased. Thus, oxidative esterification reactions described in the prior art are usually carried out at pH of about 7 to reach a tradeoff of catalyst activity and selectivity of the reaction.

The inventive process may be carried out so that a portion of the reaction mixture, preferably the one located remotely from the reactor outflow, has a pH of at most 6.5, more preferably at most 6.2, more preferably at most 6.0 during the process. The portion having the lowest pH level in the process is usually located relatively far away from the point of adding the basic solution to the mixture and/or at the points of maximal saturation of the reaction mixture with oxygen, i.e. the locations where the reaction rate may be maximal. Adjusting of this relatively low pH level in the portion of the reaction mixture lead to increased solubility of Ni, Co, Fe, Zn and/or Ti present in the compound and optionally in the egg-shell catalyst. Additionally, it also leads to decreased process selectivity to undesired 3-methoxy-2-methylpropanal and methyl 3-methoxy-2-methylpropanoate.

The inventive process is preferably carried out so that a portion of the reaction mixture, preferably the one located close to the reaction outflow, has a pH of at least 7.0, more preferably at least 7.2, more preferably at least 7.5 during the process. The portion having the highest pH level in the process is usually located at the point of adding the basic solution to the mixture. This relatively high pH level ensures high catalytic activity and lower solubility of Ni, Co, Fe, Zn and/or Ti components. Preferable location of the portion with increased pH close to the reactor outflow ensures minimizing the losses of Ni, Co, Fe, Zn and/or Ti components from the reactor in the dissolved form.

Particularly preferably, the inventive process is carried out by adding a basic solution to adjust pH of the reaction mixture in such a way that a portion of the reaction mixture preferably the one located remotely from the reaction outflow, has a pH of at most 6.5, more preferably at most 6.2, more preferably at most 6.0, while another portion of the reaction mixture, preferably the one located close to the oxidative esterification reactor outflow, has a pH of at least 7.0, more preferably at least 7.2, more preferably at least 7.5 during the process. This variant of the inventive process allows both (a) minimizing formation of undesired side products 3-methoxy-2-methylpropanal and methyl 3-methoxy-2-methylpropanoate (portion of the reaction mixture with pH≤6.5) and (b) minimizing the loss and/or replenishment of Ni, Co, Fe, Zn and/or Ti active components of the catalyst (portion of the reaction mixture with pH≥7.0).

The inventive process is carried out in the presence of the compound soluble in the reaction mixture under process conditions. Therefore, reaction mixture extracted from the oxidative esterification process may contain significant amounts of Ni, Co, Fe, Zn and/or Ti fine particles (e.g. nanoparticles) and/or ions. Such Ni, Co, Fe, Zn and/or Ti and optionally Au particles and/or ions finely dispersed and/or dissolved in the reaction mixture exiting the reactor for carrying out oxidative esterification reaction can be trapped immediately after the reactor or further in the product workup, using a suitable trapping means, such as an ion exchange resin or an appropriate adsorbent, e.g. active carbon

### Exemplary Embodiments of the Invention

### Example 1 of Catalyst Preparation (˜1wt % Au/NiO-supported on SiO₂—Al₂O₃—MgO Carrier, According to US2021047259A1)

A 250 ml beaker is initially charged with 21.36 g of Mg(NO₃)₂*6H₂O and 31.21 g of Al(NO₃)₃*9H₂O, which are dissolved in 41.85 g of demineralized water while stirring with a magnetic stirrer. Thereafter, 1.57 g of 60% HNO₃ are added while stirring. 166.67 g of silica sol (Köstrosol 1530AS from Bad Kostritz, 30% by weight of SiO₂, median size of the particles: 15 nm) are weighed into a 500 mL three-neck flask and cooled to 15° C. while stirring. 2.57 g of 60% HNO₃ are added gradually to the sol while stirring. At 15° C., the nitrate solution is added to the sol within 45 min while stirring. After the addition, the mixture is heated to 50° C. within 30 min and stirred at this temperature for a further 24 h. After this time, the mixture is spray-dried at exit temperature 130° C. The dried powder (spherical, median particle size 60 μm) is heated in a thin layer in a Naber oven to 300° C. within 2 h, kept at 300° C. for 3 h, heated to 600° C. within 2 h and finally kept at 600° C. for 3 h.

A suspension of 10 g of the SiO₂—Al₂O₃—MgO carrier from the preceding paragraph in 33.3 g of demineralized water is heated to 90° C. and stirred at this temperature for 15 min. HAuCl₄*3H₂O (205 mg) and Ni(NO₃)₂*6H₂O (567 mg, 1.95 mmol) in 8.3 g of water are heated to 90° C. and added to this suspension while stirring. After the addition, the mixture is stirred for a further 30 min, then cooled, filtered at room temperature, and subsequently washed six times with 50 ml each time of water. The material is dried at 105° C. for 10 h, finely crushed with a mortar and pestle, then heated from 18° C. up to 450° C. within 1 h and calcined at 450° C. for 5 h to yield egg-shell catalyst particles (as can be determined by EDX analysis) with a number average particle size of about 60 μm.

### Comparative Example 2 of the Prior Art Process for Oxidative Esterification of Methacrolein (According to US2021047259A1)

Gold-containing catalyst prepared as described above is put into a 200 mL stirred pressure reactor equipped with a gas-entraining stirrer. Continuous catalyst testing is carried out at the pressure of 5 bar. A feed composed of 41% by weight of methacrolein and 59% by weight of methanol (44 g/h), and also a 0.5-1.0% by weight NaOH solution in methanol (7.3 g/h), are introduced continuously into the reactor close to the stirrer to maintain pH of about 7 during the reaction. Air is utilized as oxygen source and is metered directly into the liquid reaction mixture. The offgas is cooled at −20° C. downstream of the reactor and the oxygen content therein is measured continuously. The amount of air introduced into the reactor is adjusted such that the oxygen concentration in the offgas is 4.0% by volume. After filtration, the liquid product mixture is discharged continuously from the reactor, cooled down and analyzed by means of gas chromatography (GC).

This oxidative esterification process may be continued in this way for hundreds of hours. However, due to certain catalyst attrition, especially affecting the largest catalyst particles, the active components (Au, NiO) content of the large egg-shell catalyst particles catalyst tends to decrease with time on stream and the initial egg-shell distribution of the active components tend to disappear with time leading to a more homogeneous distribution thereof throughout catalyst profile. This can in turn lead to decreased catalyst performance (methacrolein conversion and selectivity to MMA).

### Exemplary Embodiment 3 of the Process for Oxidative Esterification of Methacrolein According to the Present Invention (Ni Salt Added)

Comparative example 2 is carried out with the same catalyst and in the same way as above described, except for continuous adding a solution of 0.01 wt % Ni(NO₃)₂*6H₂O in MeOH to the reaction mixture, wherein the weight ratio of this solution to the sum of all other feeds added into the reactor (including methacrolein, methanol and NaOH) was 1:50.

Ni content of the catalyst remains constant or may even increase during the process. Presence of dissolved Ni in the reaction mixture in the form of nickel nitrate is believed to compensate the loss of NiO components from the outer region of the catalyst particles by re-depositing it from the solution. This can help to keep the catalyst structure and catalytic performance constant throughout the prolonged catalyst operation time.

### Exemplary Embodiment 4 of the Process for Oxidative Esterification of Methacrolein According to the Present Invention (Solid Ni Source Added for Providing Ni Ions in the Reaction Mixture)

Preparation of the ˜5wt % NiO-supported on SiO₂—Al₂O₃—MgO carrier compound comprising soluble nickel:

A 250 ml beaker is initially charged with 21.36 g of Mg(NO₃)₂*6H₂O and 31.21 g of Al(NO₃)₃*9H₂O, which are dissolved in 41.85 g of demineralized water while stirring with a magnetic stirrer. Thereafter, 1.57 g of 60% HNO₃ are added while stirring. 166.67 g of silica sol (Köstrosol 1530AS from Bad Kostritz, 30% by weight of SiO₂, median size of the particles: 15 nm) are weighed into a 500 mL three-neck flask and cooled to 15° C. while stirring. 2.57 g of 60% HNO₃ are added gradually to the sol while stirring. At 15° C., the nitrate solution is added to the sol within 45 min while stirring. After the addition, the mixture is heated to 50° C. within 30 min and stirred at this temperature for a further 24 h. After this time, the mixture is spray-dried at exit temperature 130° C. The dried powder (spherical, median particle size 60 μm) is heated in a thin layer in a Naber oven to 300° C. within 2 h, kept at 300° C. for 3 h, heated to 600° C. within 2 h and finally kept at 600° C. for 3 h. The calcined carrier is milled to reduce the average particle size (d₅₀) down to ˜20 μm.

A suspension of 10 g of the SiO₂—Al₂O₃—MgO support from the preceding paragraph in 33.3 g of demineralized water is heated to 90° C. and stirred at this temperature for 15 min. Solution of Ni(NO₃)₂*6H₂O (2.84 g, 9.75 mmol) in 15 g of water heated to 90° C. is added to this suspension while stirring. After the addition, the mixture is stirred for a further 30 min, then cooled, filtered at room temperature, and subsequently washed six times with 50 mL each time of water. The material is dried at 105° C. for 10 h, finely crushed with a mortar and pestle, then heated from 18° C. up to 200° C. within 1 h and kept at 200° C. for 5 h to yield ˜5wt % NiO-supported on SiO₂—Al₂O₃—MgO carrier with a number average particle size of about 20 μm comprising soluble Ni components.

Comparative example 2 is carried out in the same way as above described, except that a 0.1:1 (wt:wt) mixture of ˜5 wt % NiO-supported on SiO₂—Al₂O₃—MgO and ˜1wt % Au/NiO-supported on SiO₂—Al₂O₃—MgO is used as the compound comprising soluble Ni and the catalyst, respectively. Ni ions from the ˜5 wt % NiO-supported on SiO₂—Al₂O₃—MgO are partially dissolved in the reaction mixture during the process.

Ni content of the catalyst remains constant or may even increase during the process. Presence of dissolved Ni in the reaction mixture is believed to compensate the loss of NiO components from the outer region of the catalyst particles by re-depositing it from the solution. This can help to keep the catalyst structure and catalytic performance constant throughout the prolonged catalyst operation time.

### Exemplary Embodiment 5 of the Process for Oxidative Esterification of Methacrolein According to the Present Invention (pH Gradient in the Reaction Mixture)

Comparative example 2 is carried out with the same catalyst and in the same way as above described, except for adding NaOH solution close to the outflow section of the reactor. In this variant of the process, pH gradient is created in the reactor: the maximal pH level (of about 7.2-8.0) is adjusted in the reactor outflow section near the point of adding NaOH, leading to precipitating the metal ions on the catalyst carrier and thus preventing active components to leave the oxidative esterification reactor. The minimal pH level (in the range 5.5-6.5) is adjusted in the reactor zone with the maximal reaction rate (i.e. close to the stirrer and/or to the air entraining section). In this version of the process, Ni from the used catalyst may to some extent be soluble in the portion of the reaction mixture with particularly low pH. However, formation of some Michael addition side-products of methanol to methacrolein or MMA is greatly reduced. Thus, this process variant with pH gradient in the reactor allows reducing the amount of undesired addition products formed in the reactor while preventing losing active catalyst components and catalyst destruction.

What is claimed is:

1. Process for catalytic oxidative esterification of methacrolein with methanol and oxygen to obtain methyl methacrylate in the presence of a heterogeneous egg-shell catalyst comprising gold metal and an oxide of at least one second element selected from Ni, Co, Fe, Zn and/or Ti supported on a support material comprising SiO₂, Al₂O₃ and at least one basic element oxide, wherein the process is carried out in the presence of at least one compound, comprising Ni, Co, Fe, Zn and/or Ti, which is at least partially soluble in the reaction mixture under process conditions.

2. The process according to claim 1, wherein the maximum Ni, Co, Fe, Zn and/or Ti concentration in an outer region of the egg-shell catalyst particles extending from the surface of the particles to 40% of an equivalent diameter thereof is at least 1.2 times as high as the maximum concentration of Ni, Co, Fe, Zn and/or Ti in the inner region of the egg-shell catalyst particles existing in the remaining from the outer region of the geometric equivalent diameter.

3. The process according to claim 1, wherein the basic element is selected from alkali metals, alkaline earth metals, rare earth metals, and combinations thereof.

4. The process according to claim 1, wherein the egg-shell catalyst comprises, based on the total molar amount of gold, silicon, aluminum, the second element and the basic element: 0.03 mol % to 3 mol % of gold, 40 mol % to 90 mol % of silicon, 3 mol % to 40 mol % of aluminum, 0.1 mol % to 20 mol % of the second element and 2 mol % to 40 mol % of the basic element, and all these elements except for the gold and oxygen are present in the form of oxides.

5. The process according to claim 1, wherein the d₅₀ value of the egg-shell catalyst, determined by static light scattering (SLS) method in a 5% by weight dispersion of the catalyst in water at 25° C., is from 30 μm to 150 μm.

6. The process according to claim 1, wherein the pH of the reaction mixture in the process is adjusted by adding an alkali hydroxide solution, to achieve the pH range between 5.5 and 9.0 in the reaction mixture.

7. The process according to claim 1, wherein a portion of the reaction mixture has a pH of at most 6.5 during the process.

8. The process according to claim 1, wherein a portion of the reaction mixture has a pH of at least 7.0 during the process.

9. The process according to claim 6, wherein a portion of the reaction mixture has a pH of at most 6.5, while another portion of the reaction mixture has a pH of at least 7.0 during the process.

10. The process according to claim 1, wherein the compound comprises at least one salt of an inorganic or organic acid, and/or oxide, and/or hydroxide of the second element.

11. The process according to claim 1, wherein the compound and the egg-shell catalyst share at least one same second element.

12. The process according to claim 1, wherein the compound comprises an oxide of at least one second element selected from Ni, Co, Fe, Zn and/or Ti supported on a support material comprising SiO₂, Al₂O₃ and at least one basic element oxide.

13. The process according to claim 12, wherein d₁₀ value of the compound, determined by static light scattering (SLS) method in a 5% by weight dispersion of the compound in water at 25° C., is at most 15 μm.

14. The process according to claim 2, wherein the compound is not the egg-shell catalyst.

15. The process according to claim 1, wherein the solubility of the compound in the reaction mixture under process conditions is so that at least 1 ppm of Ni, Co, Fe, Zn and/or Ti can be detected in the filtered, homogeneous oxidative esterification reaction mixture comprising the compound using inductively coupled plasma atomic emission spectroscopy (ICP-AES) method.

16. The process according to claim 1, wherein the mass ratio of the compound to the egg-shell catalyst present in the reactor mixture during the process is in the range 0.00001 to 5.

17. The process according to claim 2, wherein the spent egg-shell catalyst, wherein the maximum Ni, Co, Fe, Zn and/or Ti concentration in an outer region of the spent egg-shell catalyst particles extending from the surface of the particles to 40% of an equivalent diameter thereof is less than 1.2 times as high as the maximum concentration of Ni, Co, Fe, Zn and/or Ti in the inner region of the spent egg-shell catalyst particles existing in the remaining from the outer region of the geometric equivalent diameter, is at least partially separated from the egg-shell catalyst, and withdrawn from the reaction mixture.

18. The process according to claim 17, wherein the spent egg-shell catalyst is separated using filtration and/or sedimentation.

19. The process according to claim 17, wherein the spent egg-shell catalyst is regenerated and returned to the oxidative esterification process, wherein the regeneration is carried out by depositing Ni, Co, Fe, Zn and/or Ti precursors and gold precursors on the outer shell of the spent egg-shell catalyst, followed by thermal decomposition of such precursors so that an egg-shell catalyst is formed.

20. The process according to claim 1, wherein Ni, Co, Fe, Zn and/or Ti particles and/or ions finely dispersed and/or dissolved in the reaction mixture exiting the reactor for carrying out oxidative esterification reaction are trapped immediately after the reactor or further in the product workup, using a suitable trapping means.
